(19) United States
(12) Patent Application Publication
(10) Pub. No.: US 2022/0371346 A1
(43) Pub. Date: Nov. 24, 2022

(54) RECORDING APPARATUS

(71) Applicant: SEIKO EPSON CORPORATION, Tokyo (JP)

(72) Inventors: Shota SAKAGAMI, MATSUMOTO-SHI (JP); Yoshikazu KOIKE, CHINO-SHI (JP)

(21) Appl. No.: 17/664,166

(22) Filed: May 19, 2022

(30) Foreign Application Priority Data
May 20, 2021 (JP) 2021-085137
May 20, 2021 (JP) 2021-085138

(51) Int. Cl. B41J 29/17; B41J 29/393

(52) U.S. Cl. CPC B41J 29/17; B41J 29/393

(57) ABSTRACT

A recording apparatus includes a recording unit configured to perform recording, an operation panel including an operation unit and a button unit configured to perform an input operation, an ultraviolet irradiation unit configured to directly irradiate the operation panel with ultraviolet rays, a sensor configured to detect presence of a person; and a controller, wherein the controller causes the ultraviolet irradiation unit to emit ultraviolet rays when the sensor detects presence of a person.

The present application is based on, and claims priority from JP Application Serial Number 2021-085137, filed May 20, 2021, and from JP Application Serial Number 2021-085138, filed May 20, 2021, the disclosures of which are hereby incorporated by reference herein in their entirety.

### BACKGROUND

### 1. Technical Field

The present disclosure relates to a recording apparatus.

### 2. Related Art

In the related art, as shown in JP-A-2002-337277, there is known a touch panel including a base material and a scratch-resistant film containing an antibacterial agent comprising a double phosphate salt containing silver.

Further, as shown in JP-A-2001-321732, there is known a cleaning mechanism for a touch panel including a protective sheet covering a surface of the touch panel, a moving mechanism for moving the protective sheet, and a cleaning means for wiping the surface of the protective sheet moved by the moving mechanism.

However, the above-described touch panel has a problem in that silver may be eluted into the environment when the touch panel is discarded, increasing the burden on the environment. In addition, the mechanism merely wipes the surface of the protective sheet with a cloth or the like, and bacteria adhering to the protective sheet cannot be sterilized, resulting in a hygienic problem.

### SUMMARY

A recording apparatus includes a recording unit configured to perform recording, an operation panel including an operation unit and a button unit configured to perform an input operation, an ultraviolet irradiation unit configured to directly irradiate the operation panel with ultraviolet rays, a sensor configured to detect presence of a person, and a controller, wherein the controller causes the ultraviolet irradiation unit to emit ultraviolet rays when the sensor detects presence of a person.

A recording apparatus includes a recording unit configured to perform recording, an operation panel including an operation unit and a button unit configured to perform an input operation, an ultraviolet irradiation unit configured to directly irradiate the operation panel with ultraviolet rays, and a controller, wherein the recording apparatus is configured to switch between a normal mode in which recording is performed and a sleep mode in which power consumption is suppressed and the controller causes the ultraviolet irradiation unit to emit ultraviolet rays when the sleep mode switches to the normal mode.

A recording apparatus includes a recording unit configured to perform recording, a touch panel having an operation surface configured to perform an input operation, a protective sheet disposed in a region including the operation surface of the touch panel, a sheet revolving unit configured to revolve the protective sheet with respect to the operation surface, a sterilization unit configured to subject the protective sheet to a sterilization treatment, a sensor configured to detect presence of a person; and a controller, wherein the controller is configured to, when the sensor detects the presence of a person, cause the protective sheet to revolve so that a part of the protective sheet that was subjected to the sterilization treatment is positioned at the operation surface.

A recording apparatus includes a recording unit configured to perform recording, a touch panel having an operation surface configured to perform an input operation, a protective sheet disposed in a region including the operation surface of the touch panel, a sheet revolving unit configured to revolve the protective sheet with respect to the operation surface, a sterilization unit configured to subject the protective sheet to a sterilization treatment, and a controller, wherein the recording apparatus is configured to switch between a normal mode in which recording is performed and a sleep mode in which power consumption is suppressed and the controller causes the protective sheet to revolve when the sleep mode switches to the normal mode.

### BRIEF DESCRIPTION OF THE DRAWINGS

FIG. 1 is a perspective view showing configuration of a recording apparatus according to a first embodiment.

FIG. 2 is a schematic diagram showing an internal structure of the recording apparatus according to the first embodiment.

FIG. 3 is a perspective view showing configuration of an operation panel according to the first embodiment.

FIG. 4 is a block diagram showing control configuration of the recording apparatus according to the first embodiment.

FIG. 5 is a flowchart showing a control method of the recording apparatus according to the first embodiment.

FIG. 6 is a flowchart showing a control method of a recording apparatus according to a second embodiment.

FIG. 7 is a perspective view showing configuration of a recording apparatus according to a third embodiment.

FIG. 8 is a schematic view showing configuration of the recording apparatus according to the third embodiment.

FIG. 9 is a perspective view showing configuration of a recording apparatus according to a fourth embodiment.

FIG. 10 is a schematic diagram showing configuration around a touch panel according to the fourth embodiment.

FIG. 11 is a schematic diagram showing configuration around the touch panel according to the fourth embodiment.

FIG. 12 is a block diagram showing control configuration of the recording apparatus according to the fourth embodiment.

FIG. 13 is a flowchart showing a control method of the recording apparatus according to the fourth embodiment.

FIG. 14 is a flowchart showing a control method of a recording apparatus according to a fifth embodiment.

FIG. 15 is a schematic diagram showing configuration of a recording apparatus according to a sixth embodiment.

### DESCRIPTION OF EMBODIMENTS

### 1. First Embodiment

First, configuration of a recording apparatus 1 will be described. The recording apparatus 1 is, for example, an ink jet printer.

In each drawing, a direction along the X axis is a depth direction of the recording apparatus 1, a direction along the Y axis is a lateral width direction of the recording apparatus 1, and a direction along the Z axis is a height direction of the recording apparatus 1. In the present embodiment, the −X direction side of the recording apparatus 1 is the apparatus front side, and the +X direction side is the apparatus rear side.

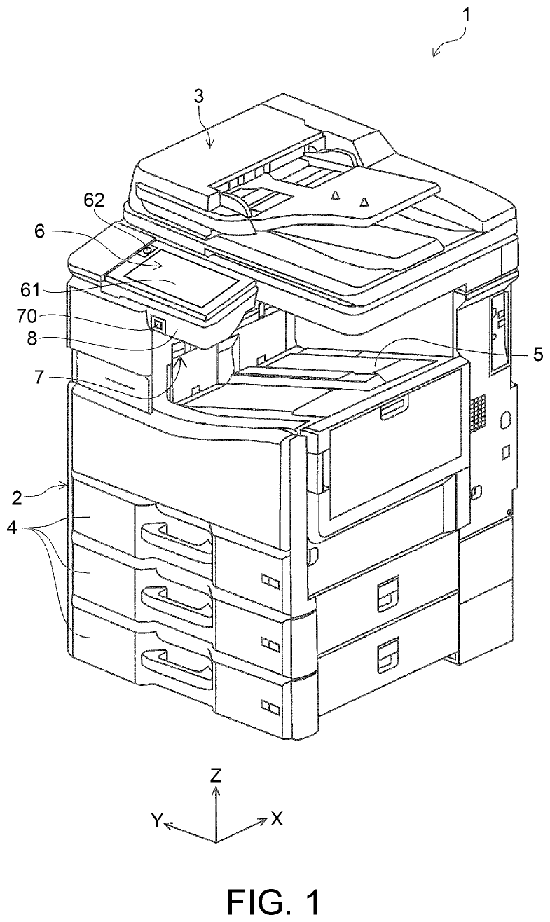

As illustrated in FIG. 1, the recording apparatus 1 according to the present embodiment is configured as a multifunction apparatus including an apparatus main body 2 and an image reading unit 3. The apparatus main body 2 includes a plurality of medium accommodation cassettes 4 that store medium P. Each of the medium accommodation cassettes 4 is detachably attached to the apparatus front surface side of the apparatus main body 2. The medium P is, for example, paper such as plain paper, thick paper, or photographic paper.

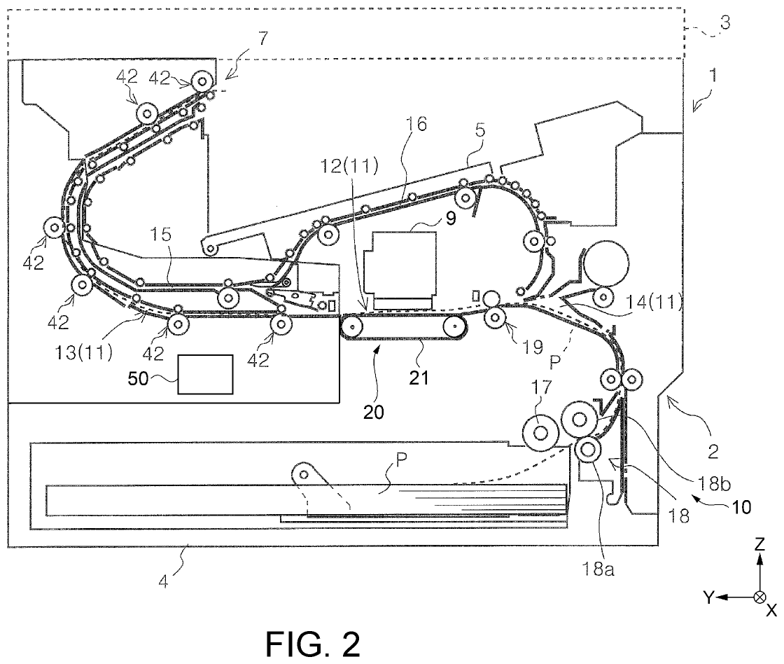

A discharge unit 7 and a medium placement unit 5 are provided between the image reading unit 3 and the medium accommodation cassettes 4 in the apparatus height direction of the apparatus main body 2. The discharge unit 7 discharges medium P on which recording was executed by a line head 9 (FIG. 2), which serves as a recording unit that performs recording by discharging ink, which is an example of a liquid. The medium placement unit 5 is where medium P discharged from the discharge unit 7 is placed.

An operation panel 6 is provided on the front side of the apparatus main body 2. The operation panel 6 includes an operation unit 61 for inputting an instruction for a recording operation, an image reading operation, or the like to the recording apparatus 1, and a button unit 62 for turning on and off the power supply of the recording apparatus 1. The operation unit 61 is, for example, a touch panel. The operation panel 6 includes a tilt mechanism, and can be used by tilting the operation panel 6, with respect to a support section 8 arranged below the operation panel 6, into an easy-to-operate position.

A sensor 70 for detecting the presence of a person is provided on the apparatus front side of the apparatus main body 2. The sensor 70 is disposed at a position where the detection position of the sensor 70 does not change. In the present embodiment, the sensor 70 is disposed at the apparatus front surface of the apparatus main body 2, at the −X direction end portion of the support section 8. This makes it possible to easily detect a person approaching the recording apparatus 1. The sensor 70 may be a thermal sensor using infrared rays, a sound wave sensor responding to sound, a sound sensitive sensor, or the like. A plurality of sensors 70 may be disposed on the apparatus main body 2. It may also be disposed at the front of the image reading unit 3.

Next, an internal structure of the recording apparatus 1 will be described.

As shown in FIG. 2, the recording apparatus 1 includes a transport path 11 for the medium P. The transport path 11 includes a feed path 14 that feeds the medium P picked up from the medium accommodation cassettes 4, a straight path 12 that is connected to the feed path 14 and that includes the recording area of the line head 9, and a face-down discharge path 13 that feeds the medium P from the straight path 12 to the discharge unit 7. The medium P is transported along the transport path 11 by a medium transport unit 10 (a feed roller 17, a separation roller pair 18, registration rollers 19, a belt conveyance unit 20, and transport roller pairs 42).

Hereinafter, transport of the medium P from the medium accommodation cassettes 4 to the discharge unit 7 will be described. Note that the recording apparatus 1 includes a switchback path 15 branched from the straight path 12 on the downstream side of the line head 9 and an inversion path 16 connected to the switchback path 15 and configured to invert the first surface (front surface) and the second surface (back surface) of the medium P and return the medium P to the straight path 12, and is configured to be capable of executing recording on a second side of the medium P after recording on a first side, that is, so called dual sided recording. Description of reversal of the medium P by the switchback path 15 and the inversion path 16 will be omitted.

In the feeding path 14, a feed roller 17 and a separation roller pair 18 that separate one sheet from a plurality of the medium P are provided in this order along the transport direction of the medium P. The feed roller 17 is configured to be rotationally driven by a drive source (not shown). The separation roller pair 18 includes a retard roller 18a serving as a drive roller that feeds the medium P toward the straight path 12 and a separation roller 18b serving as a driven roller that nips the medium P between itself and the retard roller 18a to separate the medium P.

Of the plurality of medium P accommodated in the medium accommodation cassettes 4, the uppermost medium P is picked up by the feed roller 17 and is transported downstream in the transport direction. At this time, although the medium P subsequent to the uppermost medium P may be transported together with the uppermost medium P, the uppermost medium P and the subsequent medium P are separated by the separation roller pair 18, and only the uppermost medium P is sent to the feeding path 14.

The registration rollers 19 are provided downstream of the separation roller pair 18 in the transport direction. The feeding path 14 and the straight path 12 are connected at the position of the registration rollers 19. The straight path 12 is configured as a linearly extending path, and the registration rollers 19, the belt conveyance unit 20, and the line head 9 are provided on the straight path 12. The straight path 12 is a path that passes through the recording area of the line head 9 and extends upstream and downstream from the line head 9.

In the embodiment, the belt conveyance unit 20 includes a conveyor belt 21, is disposed in a region facing the head surface of the line head 9, and supports the opposite side of the medium P than the recording surface.

The line head 9 is disposed so as to face the conveyor belt 21. When the medium P is transported to a position on the belt conveyance unit 20 facing the line head 9, the line head 9 ejects ink droplets onto the recording surface of the medium P to perform recording. The line head 9 is a recording head in which nozzles that discharge ink are provided so as to cover the entire width of the medium P, and is a recording head that is capable of recording across the entire width direction of the medium P without accompanying movement in the width direction of the medium P.

Note that the recording apparatus 1 of the present embodiment includes the line head 9, but it may be a serial type recording head that is mounted on a carriage and that performs recording by ejecting liquid onto the medium P while reciprocating in a direction intersecting the medium transport direction.

The medium P conveyed through the straight path 12 is subsequently sent to the face-down discharge path 13. The face-down discharge path 13 is a curved portion of the transport path connected to the straight path 12, and is a path for transporting the medium P so as to be discharged from the discharge unit 7 with the recording surface of the medium P recorded by the line head 9 facing downward.

The medium P that has entered the face-down discharge path 13 is transported by the plurality of transport roller pairs 42, discharged from the discharge unit 7, and placed on the medium placement unit 5 with the recording surface facing down.

Next, the configuration of the operation panel 6 will be described.

In the recording apparatus 1 are disposed ultraviolet irradiation units 80 configured to directly irradiate the operation panel 6 with ultraviolet rays.

The ultraviolet irradiation units 80 irradiate ultraviolet rays toward the operation panel 6 including the operation unit 61 and the button unit 62. As a result, the surface of the operation panel 6 where a finger touches the operation unit 61 or the button unit 62 is reliably irradiated with ultraviolet rays, and a sterilization treatment can be performed on the operation panel 6.

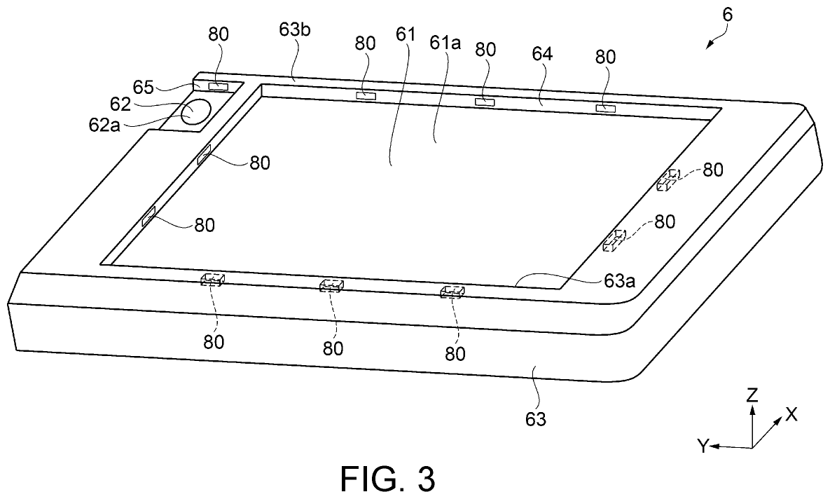

As shown in FIG. 3, the operation panel 6 of the present embodiment includes a frame body 63 having a rectangular aperture 63a. The operation unit 61 is housed in the frame body 63 in a state where an operation surface 61a for operating the operation unit 61 is exposed through the aperture 63a.

The ultraviolet irradiation units 80 of the present embodiment are disposed at edge portions of the operation panel 6. More specifically, the ultraviolet irradiation units 80 are disposed at an edge portion 64 formed by the operation surface 61a and a frame body upper surface 63b, which is the upper surface of the frame body 63. Accordingly, the structures of the recording apparatus 1 and the operation panel 6 can be made compact.

The ultraviolet irradiation units 80 have a UV-C lamp configured to emit ultraviolet rays, and the UV-C lamp is mounted in a flat casing. A slit is provided in a part of the casing, and ultraviolet rays are emitted through the slit. Ultraviolet rays spreading out from the slit in a fan-like manner are irradiated toward the operation surface 61a.

Note that the wavelengths of ultraviolet rays emitted from the UV-C lamp are 200 nm or more and 280 nm or less, and in this range, the UV-C lamp of 222 nm wavelengths is applicable.

The edge portion 64 is formed so as to surround the four sides of the operation surface 61a corresponding to the aperture 63a. A plurality of ultraviolet irradiation units 80 are disposed in the edge portion 64. In the present embodiment, the plurality of ultraviolet irradiation units 80 are arranged in four directions with respect to the operation surface 61a. As a result, the entire operation surface 61a is irradiated with ultraviolet rays, and sterilization treatment is performed on the entire operation surface 61a. The number and arrangement of the ultraviolet irradiation units 80 can be appropriately set in accordance with the size of the operation unit 61.

In addition, the button unit 62 is accommodated in the frame body 63 in a state where a top surface 62a of the button unit 62 is exposed. A step portion 65 is provided in the frame body 63 around the top surface 62a, and an ultraviolet irradiation portion 80 (UV-C lamp) for irradiating ultraviolet rays toward the top surface 62a of the button unit 62 is also disposed in the step portion 65. As a result, the top surface 62a of the button unit 62 can be subjected to sterilization treatment.

Next, a control configuration of the recording apparatus 1 will be described.

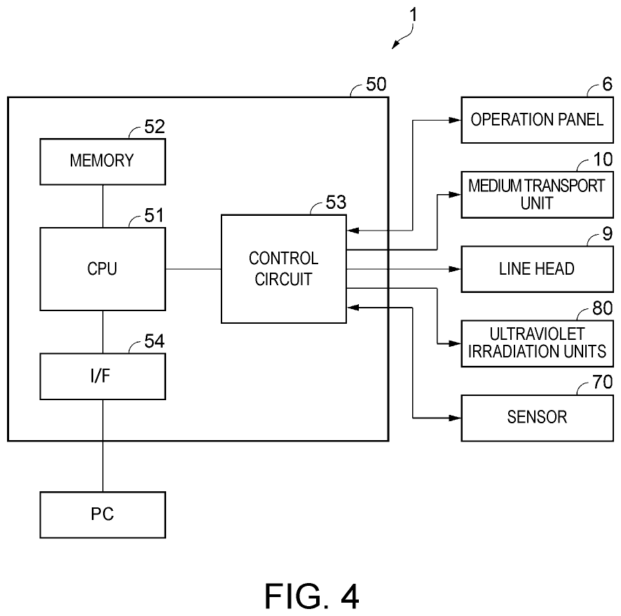

As illustrated in FIG. 4, the recording apparatus 1 includes a controller 50 that controls various operations performed by the recording apparatus 1. The controller 50 includes a CPU 51, a memory 52, a control circuit 53, and an interface (I/F) 54. The CPU 51 is an arithmetic processing unit. The memory 52 is a storage device that secures an area for storing a program of the CPU 51, a work area, or the like, and includes a storage element such as RAM and EEPROM. When recording information or the like is acquired via the I/F 54 from an external source such as an information-processing terminal (for example, a personal computer PC), the CPU 51 controls each drive unit or the like.

The controller 50 causes the ultraviolet irradiation units 80 to emit ultraviolet rays when the sensor 70 detects presence of a person. For example, when the sensor 70 detects the presence of a person, the ultraviolet irradiation units 80 irradiate ultraviolet rays for a predetermined period (for example, 10 to 20 seconds). Then, when the predetermined period has elapsed, irradiation of ultraviolet rays from the ultraviolet irradiation units 80 is stopped. By this, the operation panel 6 is directly subjected to sterilization treatment by ultraviolet rays before the user's finger touches the operation panel 6.

Also, the controller 50 causes the ultraviolet irradiation units 80 to irradiate ultraviolet rays when an input operation at the operation panel 6 is detected. For example, when an input operation is started by a finger on the operation unit 61, the contact of the finger is detected and, based on the detection result, ultraviolet rays are irradiated from the ultraviolet irradiation units 80. In addition, if finger contact is not detected over a certain period of time, ultraviolet rays are irradiated for a predetermined period of time (for example, 10 to 20 seconds). Then, when the predetermined period has elapsed, irradiation of ultraviolet rays from the ultraviolet irradiation units 80 stops. By this, when the user's finger touches the operation panel 6, the sterilization treatment of the operation panel 6 can be surely performed.

Further, the controller 50 may set an irradiation amount of ultraviolet rays emitted by the ultraviolet irradiation units 80 to be larger when a touch is detected at the operation panel 6 than an irradiation amount of ultraviolet rays emitted by the ultraviolet irradiation units 80 when the sensor 70 detects presence of a person. In this way, the operation panel 6 can be efficiently subjected to sterilization treatment by more actively irradiating ultraviolet rays when the operation panel 6 is actually touched. Note that the irradiation amount of ultraviolet rays may be regulated by the irradiation time of ultraviolet rays from the ultraviolet irradiation units 80, or may be regulated by the number of ultraviolet irradiation units 80 that are driven.

Next, a control method of the recording apparatus 1 will be described.

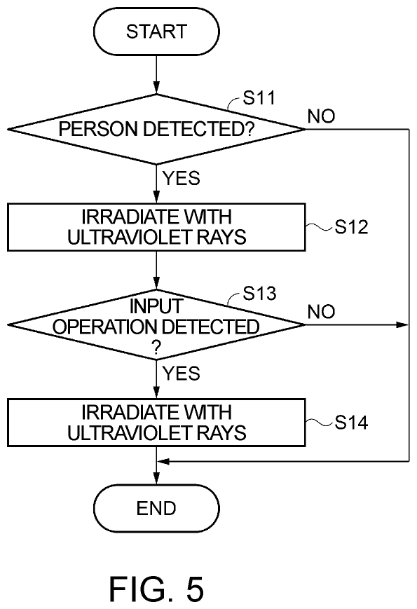

As shown in FIG. 5, in step S11, the controller 50 determines whether or not the presence of a person is detected. Specifically, it is determined whether or not the presence of a person is detected based on detection data transmitted from the sensor 70.

If the presence of a person is detected, the process proceeds to step S12, and if the presence of a person is not detected, the process ends.

In step S12, the controller 50 drives the ultraviolet irradiation units 80 to irradiate the operation panel 6 with ultraviolet rays. The ultraviolet irradiation units 80 irradiate ultraviolet rays for a predetermined period (for example, 10 to 20 seconds). Thereafter, the irradiation of ultraviolet rays from the ultraviolet irradiation units 80 is stopped.

In step S13, the controller 50 determines whether an input operation on the operation panel 6 was detected. Specifically, for example, the detection of the input operation is determined based on the detection result about contact with the operation unit 61.

If contact with the operation unit 61 is detected, the process proceeds to step S14, and if contact with the operation unit 61 is not detected, the process ends.

In step S14, the controller 50 drives the ultraviolet irradiation units 80 to irradiate the operation panel 6 with ultraviolet rays. The ultraviolet irradiation units 80 irradiate ultraviolet rays for a predetermined period (for example, 10 to 20 seconds). Thereafter, the irradiation of ultraviolet rays from the ultraviolet irradiation units 80 is stopped.

As described above, according to the present embodiment, for example, the operation panel 6 of the present embodiment does not contain silver or the like, and when the operation panel 6 is disposed of, substances such as silver are not eluted from the operation panel 6 to the environment, so the burden on the environment can be decreased.

In addition, since the operation panel 6 is subjected to sterilization treatment by ultraviolet rays before and after contact by the user, uniform sterilization can be performed on the entire operation panel, instead of localized sterilization.

In addition, since it is not necessary to perform troublesome operations with respect to the operation panel 6, such as kneading an antibacterial agent into the resin, manufacturing costs can be reduced accordingly.

### 2. Second Embodiment

Next, a second embodiment will be described.

The same configuration as that of the first embodiment is denoted by the same reference numeral, and redundant description will be omitted.

The recording apparatus 1 of the present embodiment can be switched between a normal mode in which recording is performed and a sleep mode in which power consumption is suppressed. The controller 50 causes the ultraviolet irradiation units 80 to emit ultraviolet rays when the sleep mode switches to the normal mode. The normal mode is an activated state, and the sleep mode is a standby power supply state.

The controller 50 causes the ultraviolet irradiation units 80 to emit ultraviolet rays when it is detected that the sleep mode switched to the normal mode. For example, when a print job is transmitted from the personal computer PC and the recording apparatus 1 receives the print job, the mode switches from the sleep mode to the normal mode. Then, at this timing, the ultraviolet irradiation units 80 emit ultraviolet rays for a predetermined period (for example, 10 to 20 seconds). Then, when the predetermined period has elapsed, irradiation of ultraviolet rays from the ultraviolet irradiation units 80 is stopped. By this, the operation panel 6 is directly subjected to sterilization treatment by ultraviolet rays before the user's finger touches the operation panel 6.

In addition, the controller 50 switches to the sleep mode when a predetermined time elapses after recording is performed based on the print job.

Also, the controller 50 causes the ultraviolet irradiation units 80 to irradiate ultraviolet rays when an input operation at the operation panel 6 is detected. For example, when an input operation is started by a finger on the operation unit 61, the contact of the finger is detected and, based on the detection result, ultraviolet rays are irradiated from the ultraviolet irradiation units 80. In addition, if finger contact is not detected over a certain period of time, ultraviolet rays are irradiated for a predetermined period of time (for example, 10 to 20 seconds). Then, when the predetermined period has elapsed, irradiation of ultraviolet rays from the ultraviolet irradiation units 80 stops. By this, when the user's finger touches the operation panel 6, the sterilization treatment of the operation panel 6 can be surely performed.

Further, the controller 50 may set an irradiation amount of ultraviolet rays emitted by the ultraviolet irradiation units 80 to be larger when a switch from the sleep mode to the normal mode is detected than an irradiation amount of ultraviolet rays emitted by the ultraviolet irradiation units 80 when the sensor 70 detects contact at the operation panel 6.

Next, a control method of the recording apparatus 1 according to the present embodiment will be described.

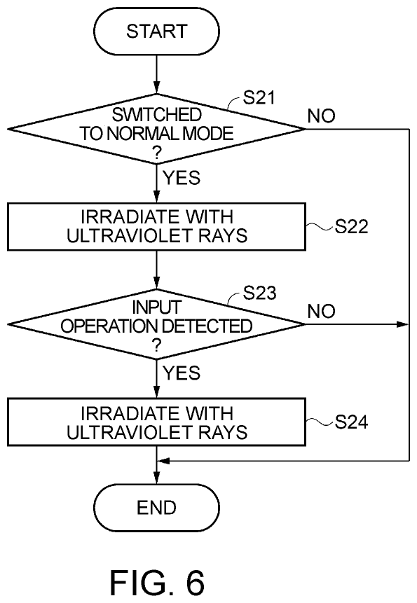

As shown in FIG. 6, in step S21, the controller 50 determines whether or not the sleep mode switched to the normal mode. Specifically, when a print job is transmitted from the personal computer PC, it is determined that the mode switched from the sleep mode to the normal mode.

When it is determined that the mode switched to the normal mode, the process proceeds to step S22, and when the mode does not switch to the normal mode (sleep mode state), the process ends.

In step S22, the controller 50 drives the ultraviolet irradiation units 80 to irradiate the operation panel 6 with ultraviolet rays. The ultraviolet irradiation units 80 irradiate ultraviolet rays for a predetermined period (for example, 10 to 20 seconds). Thereafter, the irradiation of ultraviolet rays from the ultraviolet irradiation units 80 is stopped.

In step S23, the controller 50 determines whether an input operation on the operation panel 6 was detected. Specifically, for example, the detection of the input operation is determined based on the detection result about contact with the operation unit 61.

If contact with the operation unit 61 is detected, the process proceeds to step S24, and if contact with the operation unit 61 is not detected, the process ends.

In step S24, the controller 50 drives the ultraviolet irradiation units 80 to irradiate the operation panel 6 with ultraviolet rays. The ultraviolet irradiation units 80 irradiate ultraviolet rays for a predetermined period (for example, 10 to 20 seconds). Thereafter, the irradiation of ultraviolet rays from the ultraviolet irradiation units 80 is stopped.

As described above, according to the present embodiment, when the possibility arises that a finger touches the operation panel 6, the sterilization treatment of the operation panel 6 can be reliably performed.

### 3. Third Embodiment

Next, a third embodiment will be described.

A recording apparatus 1A of the present embodiment includes ultraviolet irradiation units 80A (other ultraviolet irradiation units) at the image reading unit 3 in addition to the ultraviolet irradiation units 80 disposed at the operation panel 6. Note that the same configuration as that of the first embodiment is denoted by the same reference numeral, and redundant description will be omitted.

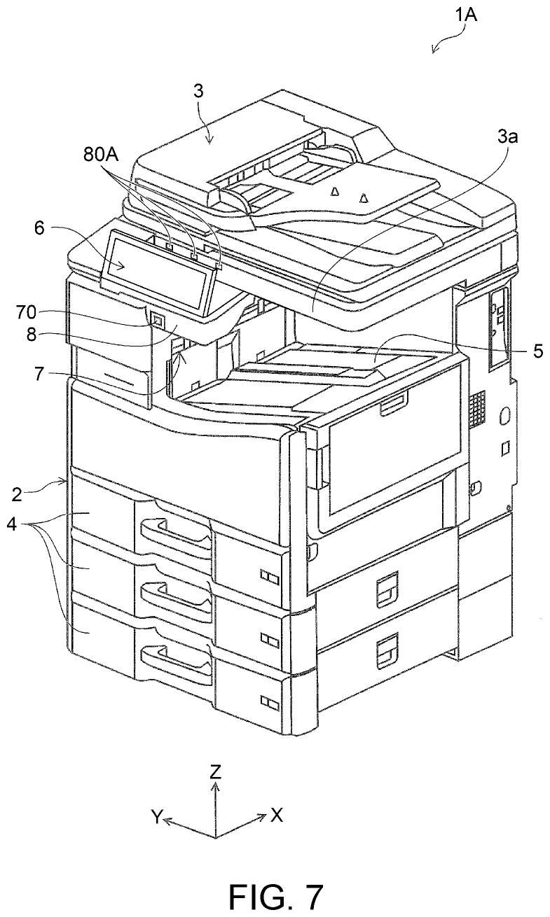

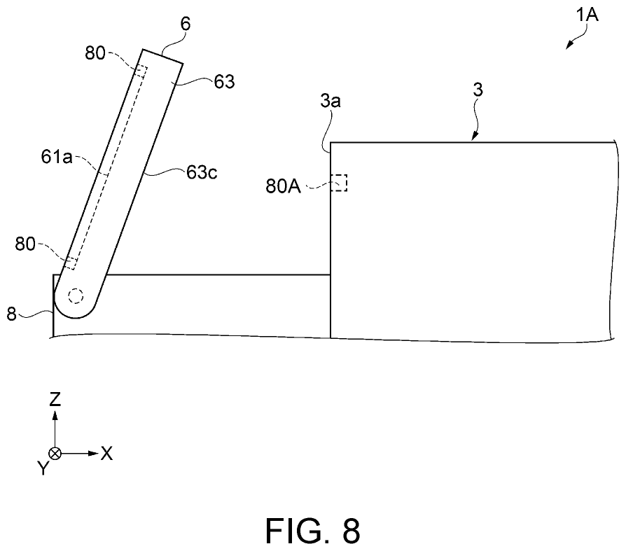

As shown in FIGS. 7 and 8, in the recording apparatus 1A of the present embodiment, an image reading unit 3 for reading an image is disposed in the vicinity of an operation panel 6, and the ultraviolet irradiation units 80A are disposed at the image reading unit 3 for irradiating the operation panel 6 with ultraviolet rays from the image reading unit 3. To be specific, the ultraviolet irradiation units 80A are disposed on the −X direction end 3a of the image reading unit 3. In the present embodiment, a plurality of sensors are arranged along the Y axis. More specifically, the ultraviolet irradiation units 80A are disposed such that, in a state in which the operation panel 6 is tilted with respect to the support section 8 by the tilt mechanism, the ultraviolet irradiation units 80A face the frame body rear surface 63c, which is the opposite surface of the operation panel 6 than the frame body upper surface 63b. The configuration of the ultraviolet irradiation units 80A is similar to that of the first embodiment, and includes a UV-C lamp.

The controller 50 causes the ultraviolet irradiation units 80, 80A to emit ultraviolet rays when the sensor 70 detects presence of a person. For example, when the sensor 70 detects the presence of a person, the ultraviolet irradiation units 80, 80A irradiate ultraviolet rays for a predetermined period (for example, 10 to 20 seconds). Then, when the predetermined period has elapsed, irradiation of ultraviolet rays from the ultraviolet irradiation units 80, 80A is stopped. By this, the operation panel 6 is directly subjected to sterilization treatment by ultraviolet rays before the user's finger touches the operation panel 6.

Also, the controller 50 causes the ultraviolet irradiation units 80,80A to irradiate ultraviolet rays when an input operation at the operation panel 6 is detected. For example, when an input operation is started by a finger on the operation unit 61, the contact of the finger is detected and, based on the detection result, ultraviolet rays are irradiated from the ultraviolet irradiation units 80, 80A. In addition, if finger contact is not detected over a certain period of time, ultraviolet rays are irradiated for a predetermined period of time (for example, 10 to 20 seconds). Then, when the predetermined period has elapsed, irradiation of ultraviolet rays from the ultraviolet irradiation units 80, 80A stops. By this, when the user's finger touches the operation panel 6, the sterilization treatment of the operation panel 6 can be surely performed.

As described above, according to this embodiment, it is possible to subject the operation surface 61a of the operation panel 6 and the frame body rear surface 63c of the operation panel 6, which are directly touched by fingers, to sterilization treatment by ultraviolet rays irradiated from the image reading unit 3 side, in addition to the ultraviolet rays irradiated by the ultraviolet irradiation units 80 disposed on the edge portion 64 of the operation panel 6. In particular, when the operation panel 6 is tilted using the tilt mechanism, sterilization treatment can be performed on the frame body rear surface 63c of the operation panel 6 which was touched by fingers.

Note that, because ultraviolet rays are irradiated from above the operation panel 6, it is possible to perform sterilization treatment on the frame body upper surface 63b of the frame body 63 of the operation panel 6 even in a state where the operation panel 6 is in contact with the support section 8 (a state in which the operation panel 6 is not inclined).

### 4. Fourth Embodiment

Next, a fourth embodiment will be described. In a recording apparatus 1B of the fourth embodiment, an operation unit 106 is provided instead of the operation panel 6 of the recording apparatus 1. Note that the same configuration as that of the above embodiments may be denoted by the same reference numeral, and redundant description will be omitted.

First, the configuration of the recording apparatus 1B will be described. The recording apparatus 1B is, for example, an ink jet printer.

In each drawing, a direction along the X axis is a depth direction of the recording apparatus 1B, a direction along the Y axis is a lateral width direction of the recording apparatus 1B, and a direction along the Z axis is a height direction of the recording apparatus 1B. In the present embodiment, the −X direction side of the recording apparatus 1B is the apparatus front side, and the +X direction side is the apparatus rear side.

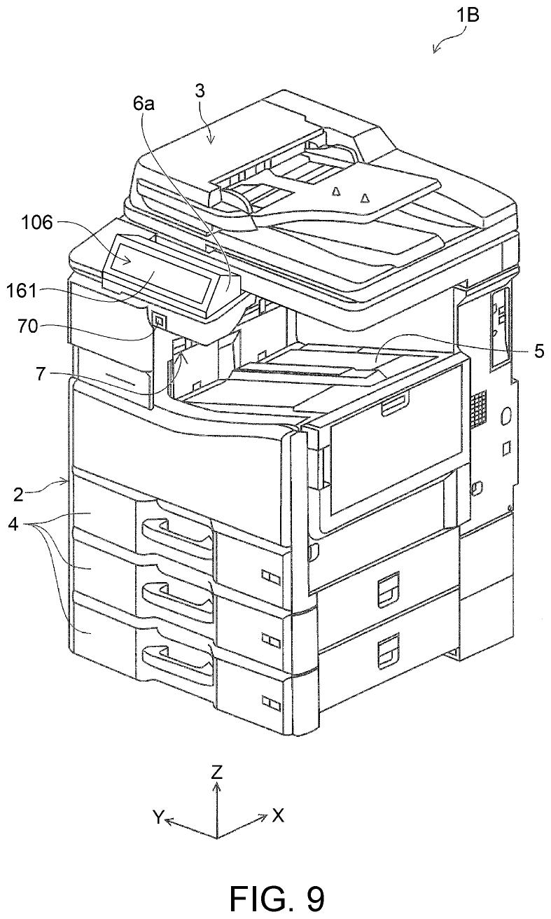

As illustrated in FIG. 9, the recording apparatus 1B according to the present exemplary embodiment is configured as a multifunction apparatus including an apparatus main body 2 and an image reading unit 3. The apparatus main body 2 includes a plurality of medium accommodation cassettes 4 that store medium P.

The operation unit 106 is provided on the front side of the apparatus main body 2. The operation unit 106 includes a touch panel 161 and the like for inputting to the recording apparatus 1B instructions for a recording operation, an image reading operation, and the like.

A sensor 70 for detecting the presence of a person is provided on the apparatus front side of the apparatus main body 2. The sensor 70 is disposed at a position where the position of the sensor 70 is fixed. In the present embodiment, the sensor 70 is a −X direction end portion of the apparatus main body 2 and is disposed below the operation unit 106. This makes it possible to easily detect a person approaching the recording apparatus 1B. The sensor 70 may be a thermal sensor using infrared rays, a sound wave sensor responding to sound, a sound sensitive sensor, or the like. A plurality of sensors 70 may be disposed on the apparatus main body 2. It may also be disposed at the front of the image reading unit 3. Since the internal structure of the recording apparatus 1B is the same as that of the above-described embodiments, description thereof will be omitted.

Next, configuration of the operation unit 106 will be described.

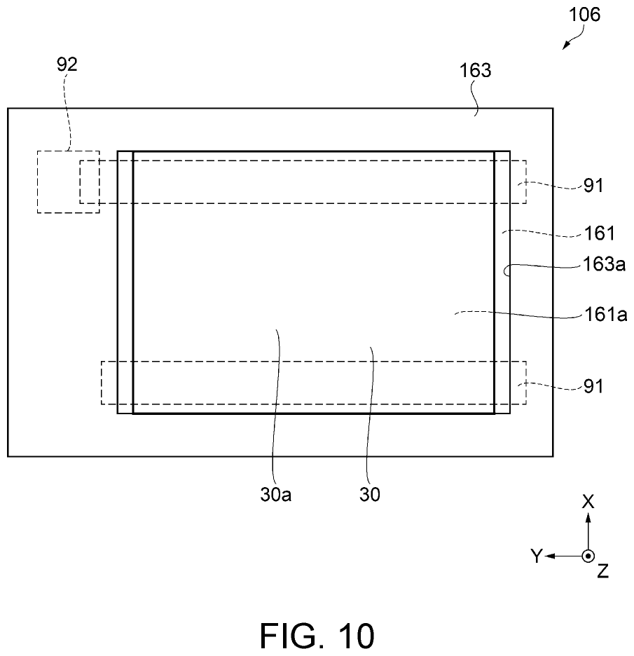

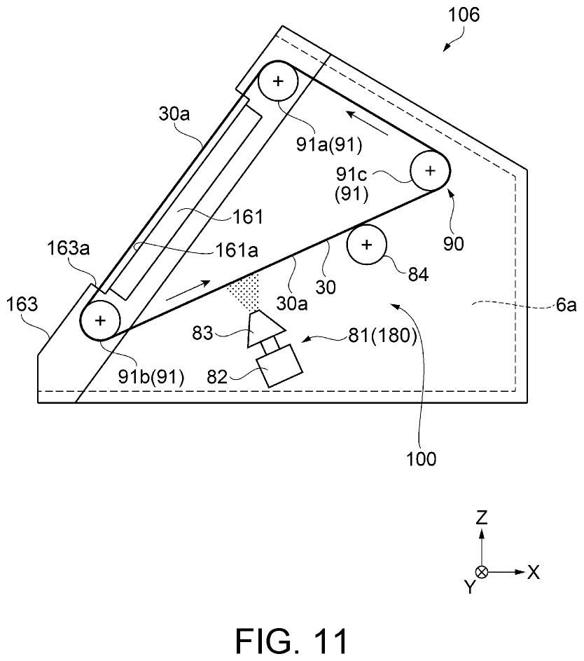

As shown in FIGS. 10 and 11, the operation unit 106 of the present embodiment includes a frame body 163 having a rectangular aperture 163a. The touch panel 161 is housed in the frame body 163 in a state in which an operation surface 161a for performing operations of the touch panel 161 can be visually checked through the aperture 163a.

In the operation unit 106 are disposed a protective sheet 30 arranged in an area including above the operation surface 161a of the touch panel 161, a sheet revolving unit 90 for revolving the protective sheet 30 with respect to the operation surface 161a, and a sterilization unit 180 for subjecting the protective sheet 30 to a sterilization treatment.

The protective sheet 30 is a transparent film having flexibility. The protective sheet 30 is disposed so as to cover the effective region of the operation surface 161a, that is, the entire region of the operation surface 161a where input operations are performed. In other words, the area that can be touched by the user's finger is covered by the protective sheet 30, and input operations of the touch panel 161 are performed through the protective sheet 30.

The sheet revolving unit 90 includes a plurality of conveying rollers 91 (91a, 91b, 91c) for revolving the protective sheet 30. Of the three conveying rollers 91, the conveying roller 91a is disposed above the upper end portion of the touch panel 161. The conveying roller 91b is disposed below a lower end portion of the touch panel 161. The conveying roller 91c is disposed in the +X direction of the conveying roller 91a. The protective sheet 30 is stretched around three conveying rollers 91a, 91b, 91c.

A gear is formed at the +Y direction end portion of the conveying roller 91a, and engages with a drive gear 92 disposed on the apparatus main body 2 side. The drive gear 92 is configured to be rotatable by a drive motor. When the drive gear 92 rotates, the conveying roller 91a rotates. The other conveying rollers 91b, 91c follow rotation of the conveying roller 91a. As a result, the protective sheet 30 revolves. In the present embodiment, the protective sheet 30 revolves from the conveying roller 91a toward the conveying roller 91b. In other words, the protective sheet 30 moves downward from above the operation surface 161a.

The sterilization unit 180 is housed in the operation unit 106. The sterilization unit 180 of the present embodiment is a sterilizing liquid spraying unit 81 configured to spray a sterilizing liquid having sterilizing capability onto the protective sheet 30. The sterilizing liquid spraying unit 81 includes a sterilizing liquid reservoir 82 for storing the sterilizing liquid, and an ejection unit 83 for ejecting the sterilizing liquid stored in the sterilizing liquid reservoir 82 toward the protective sheet 30. The ejection unit 83 is disposed on the transport path of the protective sheet 30, in between the conveying roller 91b and the conveying roller 91c. In addition, the ejection unit 83 is disposed to face a touch surface 30a of the protective sheet 30, which is touched by the user's fingers.

A plurality of nozzles are arranged in the ejection unit 83, corresponding to the width direction of the protective sheet 30, and the sterilizing liquid is applied to the entire width direction of the protective sheet 30 by discharging the sterilizing liquid from the nozzles. Thus, the touch surface 30a of the protective sheet 30 is subjected to sterilization treatment.

The sterilizing liquid is a liquid containing alcohol. Thus, the sterilization treatment can be easily performed.

The sterilizing liquid spraying unit 81 has a wiping unit 84 for wiping the sterilizing liquid applied to the protective sheet 30. The wiping unit 84 is disposed downstream of the ejection unit 83 in the transport direction of the protective sheet 30. The wiping unit 84 is, for example, a sponge roller, and is disposed in contact with the touch surface 30a of the protective sheet 30. Thus, dripping of the sterilizing liquid applied to the protective sheet 30 can be prevented. In addition, it is possible to absorb excess sterilizing liquid and promote drying of the sterilizing liquid.

The sterilizing liquid spraying unit 81 is configured to enable replenishing the sterilizing liquid reservoir 82 with sterilizing liquid. Further, the protective sheet 30 is configured to be replaceable. Specifically, in the present embodiment, the protective sheet 30, the sheet revolving unit 90, and the sterilization unit 180 are configured as a unit 100. By removing the side wall 6a of the operation unit 106, the unit 100 can be taken out from the operation unit 106. Thus, the sterilizing liquid can be easily replenished in the sterilizing liquid reservoir 82. Further, the protective sheet 30 can be replaced. For example, by replacing the protective sheet 30 before the protective sheet 30 deteriorates, it is possible to prevent the operability of the touch panel 161 from deteriorating. Note that configuration may be adopted in which a detection sensor that detects the remaining amount of the sterilizing liquid is disposed in the sterilizing liquid reservoir 82 and, based on detection information of the detection sensor, the unit 100 is taken out from the operation unit 106 and the sterilizing liquid is replenished in the sterilizing liquid reservoir 82.

Next, a control configuration of the recording apparatus 1B will be described.

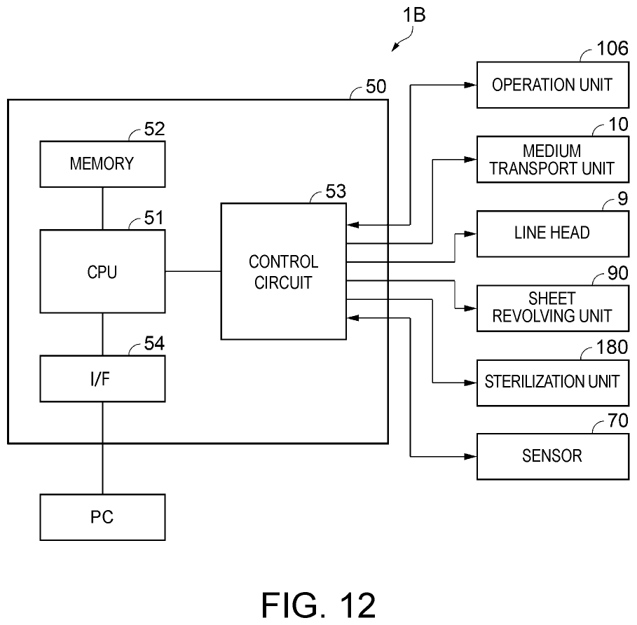

As illustrated in FIG. 12, the recording apparatus 1B includes a controller 50 that controls various operations performed by the recording apparatus 1B. The controller 50 includes a CPU 51, a memory 52, a control circuit 53, and an interface (I/F) 54. The CPU 51 is an arithmetic processing unit. The memory 52 is a storage device that secures an area for storing a program of the CPU 51, a work area, or the like, and includes a storage element such as RAM and EEPROM. When recording information or the like is acquired via the I/F 54 from an external source such as an information-processing terminal (for example, a personal computer PC), the CPU 51 controls each drive unit or the like.

When the sensor 70 detects the presence of a person, the controller 50 revolves the protective sheet 30 so that a part of the protective sheet 30 that was subjected to the sterilization treatment is positioned at the operation surface 161a. That is, the touch surface 30a of the protective sheet 30 is sterilized in advance by the sterilization unit 180 (sterilizing liquid spraying unit 81), and when the sensor 70 detects the presence of a person, the protective sheet 30 is revolved so that the portion of the protective sheet 30 that was subjected to sterilization treatment in advance is disposed above the operation surface 161a. Note that the revolving of the protective sheet 30 is regulated by a rotary encoder or the like disposed on the conveying rollers 91, and the revolving is controlled so that the portion of the protective sheet 30 subjected to the sterilization treatment is positioned above the operation surface 161a. As a result, before the user's fingers touch the protective sheet 30, the protective sheet 30 that has been subjected to sterilization treatment with the sterilizing liquid can be positioned on the operation surface 161a, and work can be performed in a sanitary environment.

Further, after detecting that the touch panel 161 is operated, the controller 50 causes the protective sheet 30 to revolve so that the portion of the protective sheet 30 subjected to the sterilization treatment is positioned above the operation surface 161a. In addition, before the protective sheet 30 revolves, sterilization treatment is performed on the portion of the protective sheet 30 positioned above the operation surface 161a. That is, when the user's fingers touch the protective sheet 30, the protective sheet 30 is revolved, and the portion of the protective sheet 30 that was subjected to sterilization treatment by the sterilizing liquid in advance is disposed above the operation surface 161a. Then, the portion of the protective sheet 30 touched by the user's fingers is subjected to sterilization treatment by being applied with sterilizing liquid. Thus, when the user's fingers touch the protective sheet 30, the portion of the protective sheet 30 touched by fingers can be reliably sterilization treated.

Further, the controller 50 may set the spray amount of sterilizing liquid sprayed by the sterilizing liquid spraying unit 81 to be larger when an operation at the touch panel 161 is detected than the spray amount of the sterilizing liquid sprayed by the sterilizing liquid spraying unit 81 when the sensor 70 detects the presence of a person. In this way, it is possible to efficiently perform sterilization treatment on the protective sheet 30 by more actively increasing the spray amount of the sterilizing liquid when the protective sheet 30 is actually touched. Note that the spraying amount of the sterilizing liquid may be regulated by the spraying amount of the sterilizing liquid per unit time, or may be regulated by the spraying time of the sterilizing liquid.

Next, a control method of the recording apparatus 1B will be described.

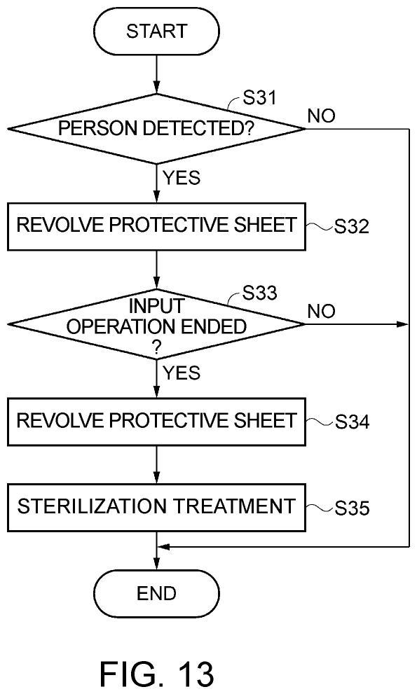

As shown in FIG. 13, in step S31, the controller 50 determines whether or not the presence of a person is detected. Specifically, it is determined whether or not the presence of a person is detected based on detection data transmitted from the sensor 70.

If the presence of a person is detected, the process proceeds to step S32, and if the presence of a person is not detected, the process ends.

When the process proceeds to step S32, the controller revolves the protective sheet 30 so that a part of the protective sheet 30 that was subjected to the sterilization treatment by sterilizing liquid is positioned above the operation surface 161a. As a result, before the user's fingers touch the protective sheet 30, the protective sheet 30 that was subjected to sterilization treatment with the sterilizing liquid can be positioned at the operation surface 161a.

In step S33, the controller 50 determines whether or not the input operation at the operation unit 106 has ended. Specifically, for example, detection of an input operation is determined based on a detection result of contact at the touch panel 161.

When contact with the touch panel 161 is detected, the process proceeds to step S34, and when contact with the touch panel 161 is not detected, the process ends.

When the process proceeds to step S34, the controller revolves the protective sheet 30 so that the part of the protective sheet 30 that was subjected to the sterilization treatment is positioned above the operation surface 161a. As a result, the portion of the protective sheet 30 touched by the user's fingers is retracted, and the portion of the protective sheet 30 that has already been subjected to sterilization treatment is disposed over the operation surface 161a.

Next, in step S35, the controller 50 executes the sterilization treatment by spraying the sterilizing liquid on the portion of the protective sheet 30 positioned over the operation surface 161a, that is, the portion touched by the user's fingers.

As described above, according to the present embodiment, since the portion of the protective sheet 30 touched by the user's fingers is reliably subjected to the sterilization treatment, a sanitary work environment can be maintained.

### 5. Fifth Embodiment

Next, a fifth embodiment will be described.

The same configuration as that of the fourth embodiment is denoted by the same reference numeral, and redundant description will be omitted.

The recording apparatus 1B of the present embodiment can be switched between a normal mode in which recording is performed and a sleep mode in which power consumption is suppressed, and the controller 50 causes the protective sheet 30 to revolve when the sleep mode switches to the normal mode. The normal mode is an activated state, and the sleep mode is a standby power supply state.

When it is detected that the sleep mode switched to the normal mode, the controller 50 uses the sheet revolving unit 90 to revolve the protective sheet 30. For example, when a print job is transmitted from the personal computer PC and the recording apparatus 1B receives the print job, the mode switches from the sleep mode to the normal mode. At this timing, the protective sheet 30 is revolved. Then, the revolving is stopped at a timing when the protective sheet 30 reaches a predetermined distance. As a result, before the user's fingers touch the protective sheet 30, the portion of the protective sheet 30 that was subjected to sterilization treatment can be positioned at the operation surface 161a.

In addition, the controller 50 switches to the sleep mode when a predetermined time elapses after recording is performed based on the print job.

Further, after detecting that the touch panel 161 is operated, the controller 50 causes the protective sheet 30 to revolve so that the portion of the protective sheet 30 subjected to the sterilization treatment is positioned above the operation surface 161a. In addition, before the protective sheet 30 revolves, sterilization treatment is performed on the portion of the protective sheet 30 positioned above the operation surface 161a. That is, when the user's fingers touch the protective sheet 30, the protective sheet 30 is revolved, and the portion of the protective sheet 30 that was subjected to sterilization treatment in advance is disposed above the operation surface 161a. Then, the portion of the protective sheet 30 touched by the user's fingers is subjected to sterilization treatment by being applied with sterilizing liquid. Thus, when the user's fingers touch the protective sheet 30, the portion of the protective sheet 30 touched by fingers can be reliably sterilization treated.

Further, the controller 50 may set the spray amount of sterilizing liquid sprayed by the sterilizing liquid spraying unit 81 to be larger when an operation at the touch panel 161 is detected than the spray amount of the sterilizing liquid sprayed by the sterilizing liquid spraying unit 81 when the sensor 70 detects the presence of a person.

Next, a control method of the recording apparatus 1B according to the present embodiment will be described.

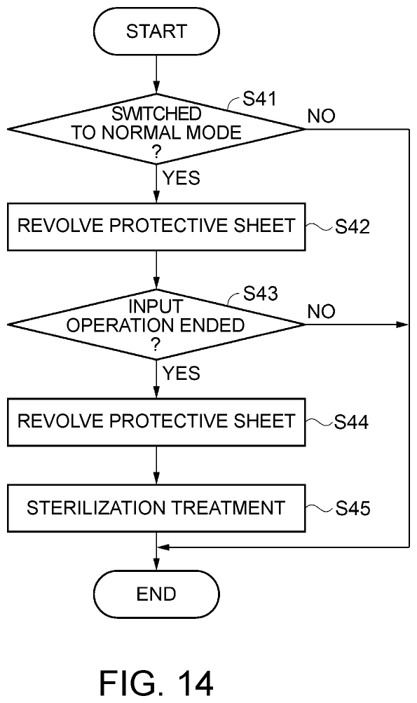

As shown in FIG. 14, in step S41, the controller 50 determines whether or not the sleep mode switched to the normal mode. Specifically, when a print job is transmitted from the personal computer PC, it is determined that the mode switched from the sleep mode to the normal mode.

When it is determined that the mode switched to the normal mode, the process proceeds to step S42, and when the mode does not switch to the normal mode (sleep mode state), the process ends.

When the process proceeds to step S42, the controller revolves the protective sheet 30 so that the part of the protective sheet 30 that was subjected to the sterilization treatment is positioned above the operation surface 161a. As a result, before the user's fingers touch the protective sheet 30, the protective sheet 30 that was subjected to sterilization treatment with the sterilizing liquid can be positioned at the operation surface 161a.

In step S43, the controller 50 determines whether or not the input operation at the operation unit 106 has ended. Specifically, for example, detection of an input operation is determined based on a detection result of contact at the touch panel 161.

When contact with the touch panel 161 is detected, the process proceeds to step S44, and when contact with the touch panel 161 is not detected, the process ends.

When the process proceeds to step S44, the controller revolves the protective sheet 30 so that the part of the protective sheet 30 that was subjected to the sterilization treatment is positioned above the operation surface 161a. As a result, the portion of the protective sheet 30 touched by the user's fingers is retracted, and the portion of the protective sheet 30 that has already been subjected to sterilization treatment is disposed over the operation surface 161a.

Next, in step S45, the controller 50 executes the sterilization treatment by spraying the sterilizing liquid on the portion of the protective sheet 30 positioned over the operation surface 161a, that is, the portion touched by the user's fingers.

As described above, according to the present embodiment, when the possibility arises that a finger touches the protective sheet 30, the sterilization treatment of the protective sheet 30 can be reliably performed.

### 6. Sixth Embodiment

Next, a sixth embodiment will be described.

In the operation unit 106 of the above-described embodiment, the sterilization unit 180 is described as the sterilizing liquid spraying unit 81, but the sterilization unit 180 is not limited the sterilizing liquid spraying unit 81. In the operation unit 206 of the present embodiment, the sterilization unit 180 includes ultraviolet irradiation units 85. Note that the same configuration as that of the fourth embodiment is denoted by the same reference numeral, and redundant description will be omitted.

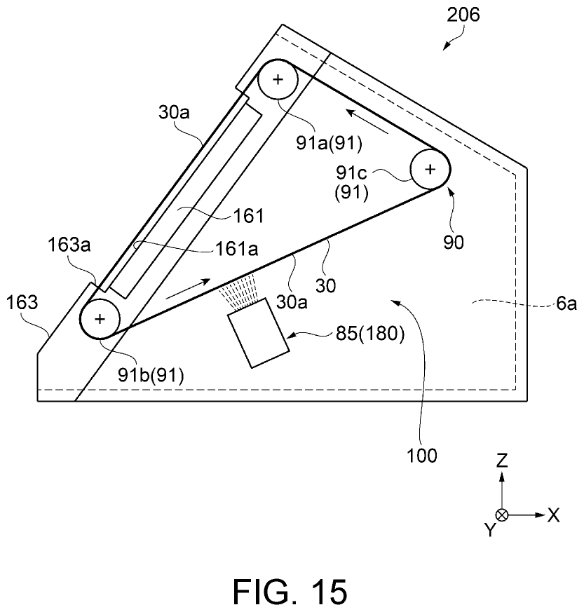

As shown in FIG. 15, the ultraviolet irradiation units 85 are disposed on the transport path of the protective sheet 30, in between the conveying roller 91b and the conveying roller 91c. In addition, the ultraviolet irradiation units 85 are disposed to face a touch surface 30a of the protective sheet 30, which is touched by the user's fingers.

The ultraviolet irradiation units 85 have a UV-C lamp configured to emit ultraviolet rays, and the UV-C lamp is mounted in a flat casing. A slit is provided in a part of the casing, and ultraviolet rays are emitted through the slit. Ultraviolet rays spreading out from the slit in a fan-like manner are irradiated on the touch surface 30a. A plurality of ultraviolet irradiation units 85 are arranged corresponding to the width direction of the protective sheet 30. Note that the wavelengths of ultraviolet rays emitted from the UV-C lamp are 200 nm or more and 280 nm or less, and in this range, the UV-C lamp of 222 nm wavelengths is applicable.

When the sensor 70 detects the presence of a person, the controller 50 revolves the protective sheet 30 so that a part of the protective sheet 30 that was subjected to the sterilization treatment is positioned at the operation surface 161a. That is, the touch surface 30a of the protective sheet 30 is sterilized in advance by the ultraviolet irradiation units 85, and when the sensor 70 detects the presence of a person, the protective sheet 30 is revolved so that the portion of the protective sheet 30 that was subjected to sterilization treatment in advance is disposed above the operation surface 161a. As a result, before the user's fingers touch the protective sheet 30, the protective sheet 30 that has been subjected to sterilization treatment by irradiation with ultraviolet rays can be positioned on the operation surface 161a, and work can be performed in a sanitary environment.

Further, after detecting that the touch panel 161 was operated, the controller 50 causes the protective sheet 30 to revolve so that the portion of the protective sheet 30 subjected to the sterilization treatment is positioned above the operation surface 161a. In addition, before the protective sheet 30 revolves, sterilization treatment is performed on the portion of the protective sheet 30 positioned above the operation surface 161a. That is, when the user's fingers touch the protective sheet 30, the protective sheet 30 is revolved, and the portion of the protective sheet 30 that has been subjected to sterilization treatment by ultraviolet irradiation in advance is disposed above the operation surface 161a. Then, the portion of the protective sheet 30 touched by the user's fingers is irradiated by ultraviolet rays from the ultraviolet irradiation units 85 for a predetermined period (for example, 10 to 20 seconds). Then, when the predetermined period has elapsed, irradiation of ultraviolet rays from the ultraviolet irradiation units 85 is stopped. Thus, when the user's fingers touch the protective sheet 30, the portion of the protective sheet 30 touched by fingers can be reliably sterilization treated.

Further, the controller 50 may set an irradiation amount of ultraviolet rays emitted by the ultraviolet irradiation units 85 to be larger when an operation is detected at the touch panel 161 than an irradiation amount of ultraviolet rays emitted by the ultraviolet irradiation units 85 when the sensor 70 detects presence of a person. In this way, the operation unit 206 can be efficiently subjected to sterilization treatment by more actively irradiating ultraviolet rays when the operation unit 206 is actually touched. Note that the irradiation amount of ultraviolet rays may be regulated by the irradiation time of ultraviolet rays from the ultraviolet irradiation units 85, or may be regulated by the number of ultraviolet irradiation units 85 that are driven.

As described above, according to the present embodiment, since the portion of the protective sheet 30 touched by the user's fingers is reliably subjected to the sterilization treatment, a sanitary work environment can be maintained.

In addition, the sterilizing liquid spraying unit 81 and the ultraviolet irradiation units 85 may be disposed as the sterilization unit 180, and the sterilizing liquid spraying unit 81 and the ultraviolet irradiation units 85 may be used together.

What is claimed is:

1. A recording apparatus comprising:
a recording unit configured to perform recording;
an operation panel including an operation unit and a button unit configured to perform an input operation;
an ultraviolet irradiation unit configured to directly irradiate the operation panel with ultraviolet rays;
a sensor configured to detect presence of a person; and
a controller, wherein
the controller causes the ultraviolet irradiation unit to emit ultraviolet rays when the sensor detects presence of a person.

2. The recording apparatus according to claim 1, wherein
the controller causes the ultraviolet irradiation unit to emit ultraviolet rays when an input operation at the operation panel is detected.

3. The recording apparatus according to claim 1, wherein
the controller sets an irradiation amount of ultraviolet rays emitted by the ultraviolet irradiation unit to be larger when a touch is detected at the operation panel than an irradiation amount of ultraviolet rays emitted by the ultraviolet irradiation unit when the sensor detects presence of a person.

4. A recording apparatus comprising:
a recording unit configured to perform recording;
an operation panel including an operation unit and a button unit configured to perform an input operation;
an ultraviolet irradiation unit configured to directly irradiate the operation panel with ultraviolet rays; and
a controller, wherein
the recording apparatus is configured to switch between a normal mode in which recording is performed and a sleep mode in which power consumption is suppressed and
the controller causes the ultraviolet irradiation unit to emit ultraviolet rays when the sleep mode switches to the normal mode.

5. The recording apparatus according to claim 1, wherein
the ultraviolet irradiation unit emits ultraviolet rays toward the surface of the operation panel.

6. The recording apparatus according to claim 5, wherein
the ultraviolet irradiation unit is disposed at an edge portion of the operation panel.

7. The recording apparatus according to claim 1, wherein
the ultraviolet irradiation unit includes a UV-C lamp that emits ultraviolet rays.

8. The recording apparatus according to claim 1, wherein
an image reading unit configured to read an image is disposed in the vicinity of the operation panel and
another ultraviolet irradiation unit configured to irradiate the operation panel with ultraviolet rays from the image reading unit is arranged at the image reading unit.

9. A recording apparatus comprising:
a recording unit configured to perform recording;
a touch panel having an operation surface configured to perform an input operation;
a protective sheet disposed in a region including the operation surface of the touch panel;
a sheet revolving unit configured to revolve the protective sheet with respect to the operation surface;
a sterilization unit configured to subject the protective sheet to a sterilization treatment;
a sensor configured to detect presence of a person; and
a controller, wherein
the controller is configured to
when the sensor detects the presence of a person, cause the protective sheet to revolve so that a part of the protective sheet that was subjected to the sterilization treatment is positioned at the operation surface.

10. The recording apparatus according to claim 9, wherein
the recording apparatus is configured to switch between a normal mode in which recording is performed and a sleep mode in which power consumption is suppressed and
the controller causes the protective sheet to revolve when the sleep mode switches to the normal mode.

11. The recording apparatus according to claim 9, wherein
after an operation at the touch panel is detected, the controller causes the protective sheet to revolve so that a portion of the protective sheet that was subjected to the sterilization treatment is positioned at the operation surface and executes the sterilization treatment on the portion of the protective sheet that was positioned at the operation surface before revolving the protective sheet.

12. The recording apparatus according to claim 9, wherein
the sterilization unit is a sterilizing liquid spraying unit configured to spray a sterilizing liquid having sterilizing capability onto the protective sheet.

13. The recording apparatus according to claim 12, wherein
the sterilizing liquid spraying unit includes a wiping unit configured to wipe the sterilizing liquid.
